(19) United States
(12) Patent Application Publication
(10) Pub. No.: US 2017/0176475 A1
(43) Pub. Date: Jun. 22, 2017

(54) NOVEL METHODS AND KITS FOR DETECTING A RIFAMYCIN, OR DERIVATIVE OR ANALOGUE THEREOF

(71) Applicant: DREXEL UNIVERSITY, Philadelphia, PA (US)

(72) Inventors: Christopher VINNARD, Pennington, NJ (US); Joseph M. SALVINO, Chester Springs, PA (US); Hans P. SCHLECHT, Philadelphia, PA (US)

(21) Appl. No.: 15/302,054

(22) PCT Filed: Apr. 9, 2015
(86) PCT No.: PCT/US15/25097

§ 371 Date: Oct. 5, 2016

Related U.S. Application Data
(60) Provisional application No. 61/978,399, filed on Apr. 11, 2014.

(51) Int. Cl. G01N 33/94

(52) U.S. Cl. CPC G01N 33/9446; G01N 2800/52

(57) ABSTRACT

The present invention includes methods of identifying one or more rifamycins, or analogues or metabolites thereof, that are present in a biological sample from a subject that is being administered a rifamycin-containing medication. The present invention further includes methods of adjusting and/or optimizing the dosage regime for a rifamycin-containing medication.

### CROSS-REFERENCE TO RELATED APPLICATIONS

The present application claims priority under 35 U.S.C. §119(e) to U.S. Provisional Application No. 61/978,399, filed Apr. 11, 2014, which is hereby incorporated by reference in its entirety herein.

### STATEMENT REGARDING FEDERALLY SPONSORED RESEARCH OR DEVELOPMENT

This invention was made with government support under Contract No. R21AI104441-01 awarded by the National Institute of Allergy and Infectious Diseases (NIAID/NIH). The government has certain rights in the invention.

### BACKGROUND OF THE INVENTION

The rifamycins are a class of bactericidal antibiotics that bind tightly to prokaryotic RNA polymerase, inhibiting DNA-dependent RNA synthesis. The rifamycins bind to RNA polymerase at a site adjacent to the enzyme's active center, and block RNA synthesis by physically preventing extension of RNA products beyond a length of 2-3 nucleotides (generally referred to as “steric-occlusion” mechanism). These drugs are used as first line treatment for tuberculosis (Tb), especially HIV-related Tb.

The best characterized rifamycin antibiotic is rifampicin, also known as rifampin. Rifampicin rapidly kills fast-dividing bacilli strains, acting as a potent bactericide. Rifampicin is typically used to treat Mycobacterium infections, including tuberculosis and leprosy (Hansen's disease). It can also be used to treat abscesses derived from an uncommon complication of BCG vaccination for tuberculosis.

Rifampicin is used in the treatment of methicillin-resistant Staphylococcus aureus (MRSA) in combination with fusidic acid, such as in osteomyelitis and prosthetic joint infections. It is also used in prophylactic therapy against Neisseria meningitidis (meningococcal) infection. Rifampicin is recommended as an alternative treatment for infections with the tick-borne disease pathogens Borrelia burgdorferi and Anaplasma phagocytophilum when treatment with doxycycline is contraindicated (e.g., in pregnant women or patients with a history of allergy to tetracycline antibiotics). Rifampicin is further used to treat infection by Listeria species, Neisseria gonorrhoeae, Haemophilus influenzae and Legionella pneumophila.

Rifampicin is a major component of the “PIERS” cocktail-drug treatment of tuberculosis and inactive meningitis, along with pyrazinamide, isoniazid, ethambutol and streptomycin. Rifampicin must be administered regularly daily for several months without break; otherwise, the risk of development of drug-resistant tuberculosis is greatly increased. In fact, the presence of rifampicin in the PIERS cocktail is the primary motivation behind the directly observed therapy for tuberculosis.

Orally administered rifampicin is readily absorbed through the gastrointestinal tract, resulting in peak plasma drug concentrations in about two to four hours after intake. Interestingly, peak serum concentrations for rifampicin vary widely from individual to individual. For example, HIV-infected patients have decreased rifampicin circulating concentrations, compared with HIV-uninfected patients. Rifampicin is rapidly eliminated in the bile and undergoes progressive entrohepatic circulation and deacetylation to the primary metabolite 25-desacetyl-rifampicin. In normal metabolizing patients, nearly all the drug in the bile is present in the deacetylated form within about 6 hours. Deacetylated rifamycin is still a potent antibiotic, but no longer reabsorbable by the intestine, being subsequently eliminated from the body. Typically, 30% of a rifamycin dose is excreted in the urine, with about half of this being unchanged drug.

Additionally, about 30% of the treated population over-metabolize rifamycin to its deacetylated metabolite. When administered to these over-metabolizing individuals, rifamycin is quickly hydrolyzed to its deacetylated metabolite, which is excreted in urine. As such, the over-metabolizing individuals have an overall suboptimal exposure to rifamycin, resulting in poor efficacy of the antibiotic treatment.

There is a need in the art for novel methods of detecting a rifamycin and/or its metabolites or analogues in a biological sample, such as urine, from an individual being administered a rifamycin-containing medication. The present invention addresses and meets these needs.

### BRIEF SUMMARY OF THE INVENTION

The invention provides a method of detecting the presence of a rifamycin, or metabolite or analogue thereof, in a sample. The invention further provides a method of determining whether a subject is being administered an effective dosage of a rifamycin-containing medication. The invention further provides a kit.

In certain embodiments, the method comprises contacting the sample with a derivatization reagent comprising an activated carboxylic acid, using conditions under which at least one phenolic group in the rifamycin, or metabolite or analogue thereof, reacts with the activated carboxylic acid to form an ester-comprising rifamycin derivative, thereby generating a reaction mixture. In other embodiments, the method comprises analyzing the reaction mixture for the presence of the ester-comprising rifamycin derivative. In yet other embodiments, if the derivative is present in the reaction mixture, the rifamycin, or metabolite or analogue thereof, is detected in the sample. In yet other embodiments, the method comprises contacting the derivatization reagent with at least one control sample comprising a known amount of the rifamycin, or metabolite or analogue thereof; thereby generating a control mixture. In yet other embodiments, the method comprises analyzing the control mixture for the presence of the ester-comprising rifamycin derivative. In yet other embodiments, the method comprises quantitating the amount or concentration of the rifamycin, or metabolite or analogue thereof, in the sample.

In certain embodiments, the sample comprises a biological sample from a subject that is administered a rifamycin-containing medication. In other embodiments, the sample comprises urine, blood or saliva from the subject. In yet other embodiments, the derivatization reagent comprises a chromophore. In yet other embodiments, the chromophore is UV-active or fluorescent. In yet other embodiments, the rifamycin-containing medication comprises at least one selected from the group consisting of rifampicin, rifamycin B, rifamycin SV, rifamycin S, rifabutin, rifapentine, rifaximin, and metabolites and analogues thereof. In yet other embodiments, the rifamycin is present in the rifamycin-containing medication. In yet other embodiments, the rifamycin metabolite comprises the deacetylated derivative of the rifamycin-containing medication. In yet other embodiments, the sample is obtained from the subject at a given period of time after the subject is administered the rifamycin-containing medication.

In certain embodiments, the activated carboxylic acid is selected from the group consisting of acyl chloride, symmetric or mixed acid anhydride, vinyl ester, cyanomethyl ester, S-phenyl thioester, piperidino ester, pyrid-3-yl ester, p-nitrophenyl ester, 2,4,6-trichlorophenyl ester, 2,3,4,5,6-pentachlorophenyl ester, tetrafluorophneyl ester, 2,3,4,5,6-pentafluorophenyl ester, phtalimido ester, succinimido ester, 4-oxo-3,4-dihydrobenzotriazin-3-yl ester, and benzotriazolyl ester. In other embodiments, the derivatization reagent is immobilized on a solid support.

In certain embodiments, the analysis of the control mixture allows for the generation of a calibration curve for the rifamycin, or metabolite or analogue thereof. In certain embodiments, the subject is human.

In certain embodiments, the method comprises contacting a biological sample from the subject with a derivatization reagent comprising an activated carboxylic acid, using conditions under which at least one phenolic group in the rifamycin, or metabolite or analogue thereof, reacts with the activated carboxylic acid to form an ester-comprising rifamycin derivative, thereby generating a reaction mixture. In other embodiments, the method comprises analyzing the reaction mixture for the presence of the ester-comprising rifamycin derivative. In yet other embodiments, the method comprises determining the concentration of the rifamycin, or metabolite or analogue thereof in the biological sample. In yet other embodiments, the method comprises determining the circulating concentration of the rifamycin, or metabolite or analogue thereof, in the subject. In yet other embodiments, the method comprises determining whether the subject is being administered an effective dosage of the rifamycin-containing medication. In yet other embodiments, the method comprises adjusting the subject's dosage of the rifamycin-containing medication so that a therapeutically effective rifamycin circulating concentration is reached in the subject.

In certain embodiments, the sample comprises urine, blood or saliva from the subject. In other embodiments, the derivatization reagent comprises a chromophore. In yet other embodiments, the chromophore is UV-active or fluorescent. In yet other embodiments, the rifamycin-containing medication comprises at least one selected from the group consisting of rifampicin, rifamycin B, rifamycin SV, rifamycin S, rifabutin, rifapentine, rifaximin, and metabolites and analogues thereof. In yet other embodiments, the rifamycin is present in the rifamycin-containing medication. In yet other embodiments, the rifamycin metabolite comprises the deacetylated derivative of the rifamycin-containing medication. In other embodiments, the sample is obtained from the subject at a given period of time after the subject is administered the rifamycin-containing medication. In yet other embodiments, the subject is human.

In certain embodiments, the kit comprises an activated carboxylic acid, wherein the activated carboxylic acid reacts with at least one phenolic group in a rifamycin, or metabolite or analogue thereof, and instructional material comprising instructions how to detect the rifamycin, or metabolite or analogue thereof, using the activated carboxylic acid as a reagent.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic representation of the derivatization of rifampicin and its deacetylated derivative with an UV-active fluorescent dye comprising a tether and an activated carboxylic acid (in this case, an acyl chloride). In the non-limiting examples of derivatizing agents, X can be halide, hydroxysuccinate, p-nitrophenoxide, mixed anhydride, or the like; Z can be —O—, —C(═O)NH—, —NH(C═O)—, —S(═O)₂NH—, —NHS(═O)₂—, or the like; Dye can be classic organic dye, AlexFluor coumarin dye, quantum dot, or the like.

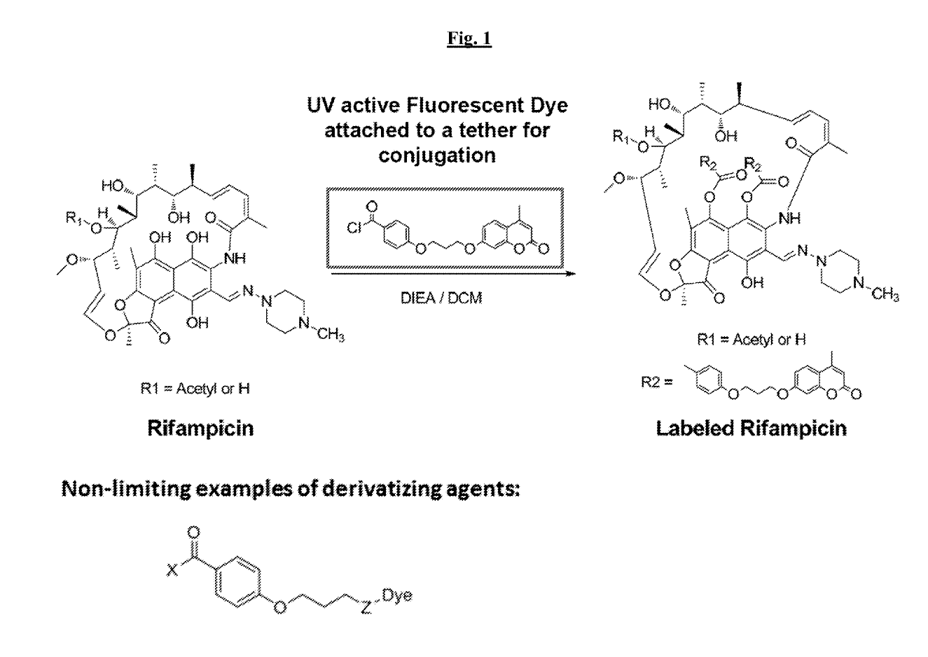

FIG. 2 is a schematic representation of the reaction of rifampicin and its deacetylated derivative with a solid-phase reagent comprising an UV-active fluorescent dye tethered to the solid particle through an activated carboxylic ester. In this reaction, rifampicin and its deacetylated derivative are labeled with the UV-active fluorescent dye.

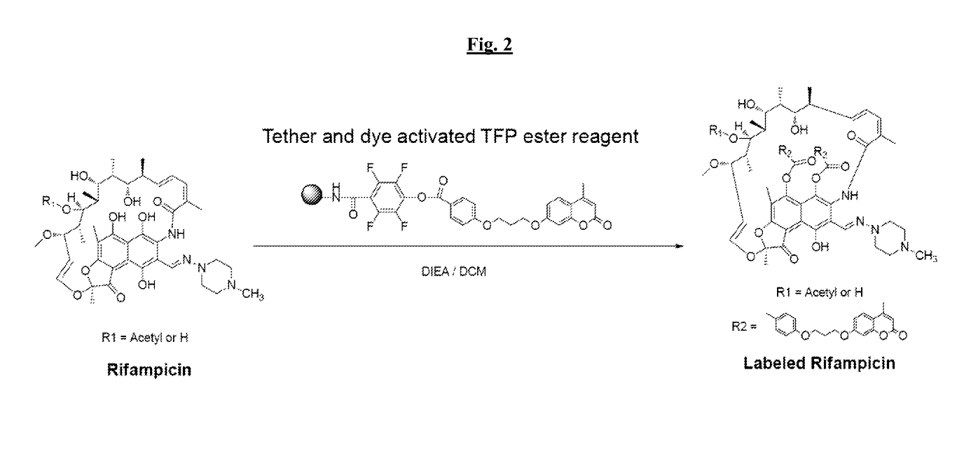

FIG. 3 is a schematic representation of the possible sites of rifabutin and rifapentine that may be derivatized as an ester under the reaction conditions contemplated within the invention.

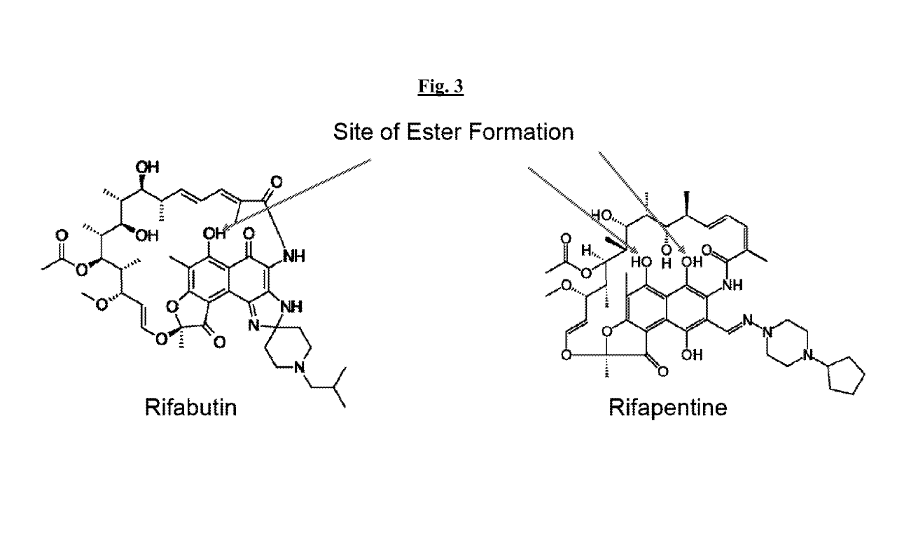

### DETAILED DESCRIPTION OF THE INVENTION

The present invention relates to the unexpected discovery of a versatile method of identifying one or more rifamycins, or analogues or metabolites thereof, that are present in a sample. The present invention further includes a method of identifying one or more rifamycins, or analogues or metabolites thereof, that are present in a biological sample obtained from a subject that is being administered a rifamycin-containing medication. In one aspect, the biological sample comprises urine, blood or saliva.

In certain embodiments, the methods of the invention allow the determination of the rate and/or extent of in vivo conversion of the administered rifamycin to its corresponding metabolite(s). In other embodiments, the methods of the invention allow the identification of individuals that over-metabolize the drug, as well as adjust and/or optimize the dosage regime of the rifamycin-containing medication so that the individual can receive an effective dose of medication.

According to the invention, the biological sample, or a sample that is obtained by manipulation of the biological sample, is contacted with a reagent comprising a chromophore. In certain embodiments of the invention, the chromophore is ultraviolet (UV)-active and/or fluorescent. In other embodiments of the invention, the reagent is selected so that it reacts with a rifamycin, or metabolite or analogue thereof, wherein the chromophore is covalently attached through a linker to at least one of the phenolic groups in the rifamycin, or metabolite or analogue thereof. The resulting chromophore-labeled rifamycin derivative may then be analyzed using an analytical method known to those in the art.

Current methods of analysis of rifamycin drugs, or metabolites or analogues thereof, in biological samples require sophisticated analytical equipment and highly trained technicians, both of which are unable in low-resource settings with high burden of tuberculosis disease. In one aspect, the methods of the present invention represent a practical approach that is suitable for use in a resource-limited environment and a developing country, where ready access to technology or medical training is limited.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “applicator” refers to any device including, but not limited to, a hypodermic syringe, a pipette, an automatic sample probe and the like, for administering and/or manipulating the compounds and compositions of the invention.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, an “effective amount,” “therapeutically effective amount” or “pharmaceutically effective amount” of a compound is that amount of compound that is sufficient to provide a beneficial effect to the subject to which the compound is administered.

As used herein, the term “instructional material” includes a publication, a recording, a diagram, a product insert or any other medium of expression that may be used to communicate the usefulness of the composition, compound and/or method of the invention in the kit. Optionally, or alternately, the instructional material may describe one or more methods related to the present invention, including as disclosed elsewhere herein.

The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Alternately, the instructional material may be obtained on the Internet in a format suitable for electronic file transmission to the user. For example, the instructional material is for use of a kit; instructions for use of the compound; or instructions for use of a formulation of the compound.

As used herein, the terms “manipulate” or “manipulation” refers to any physical and/or chemical processes that a sample may be submitted to, such as but not limited to filtration, degassing, concentration, centrifugation, dilution with a liquid, fractional precipitation, extraction with a solvent, contacting with a solid (such as a resin comprising an immobilized reagent), and the like.

As used herein, the term “metabolite” refers to a degradation and/or derivatization product of a compound that is formed in vivo upon administration of the compound to a subject. In certain embodiments of the invention, a metabolite of a rifamycin comprises the deacetylated product of a rifamycin, wherein the deacetylated product may or not be further degraded or derivatized as compared to the parent rifamycin.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

As used herein, the term “rifamycin” refers to a member of the rifamycin class of drugs. Non-limiting examples of rifamycins include:

rifampicin or rifampin, also known as (7S,9E,11S,12R,13S,14R,15R,16R,17S, 18S,19E,21Z)-2,15,17,27,29-pentahydroxy-11-methoxy-3,7,12,14,16,18,22-heptamethyl-26-{(E)-[(4-methylpiperazin-1-yl)imino]methyl}-6,23-dioxo-8,30-dioxa-24-azatetracyclo [23.3.1.1^{4,7}.0^{5,28}]triaconta-1(28),2,4,9,19,21,25(29),26-octaen-13-yl acetate:

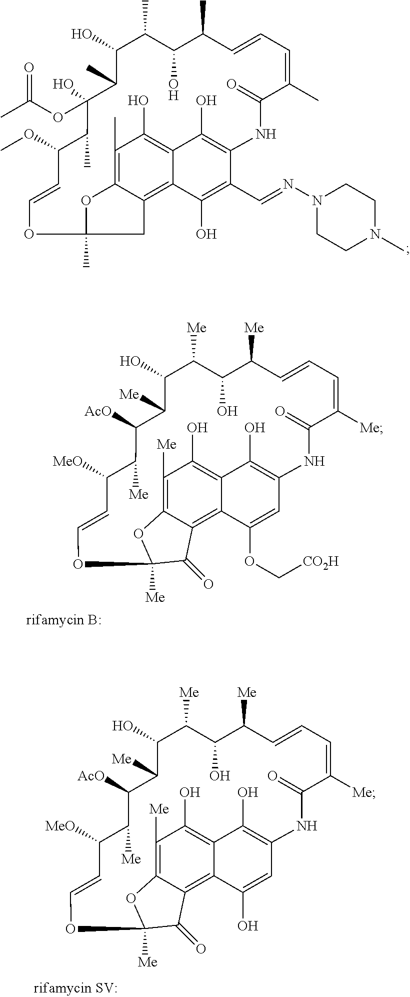

rifamycin S (wherein the 1,4-hydroxyphenyl ring in rifamycin SV is oxidized to the corresponding 1,4-quinone);

rifabutin, also known as (9S,12E,14S,15R,16S,17R,18R,19R,20S,21S,22E,24Z)-6,16,18,20-tetrahydroxy-1′-isobutyl-14-methoxy-7,9,15,17,19,21,25-hepta-methyl-spiro[9,4-(epoxypentadeca[1,11,13]trienimino)-2H-furo-[2′,3′:7,8]-naphth[1,2-d]imidazol-2,4′-piperidin]-5,10,26-(3H,9H)-trione-16-acetate:

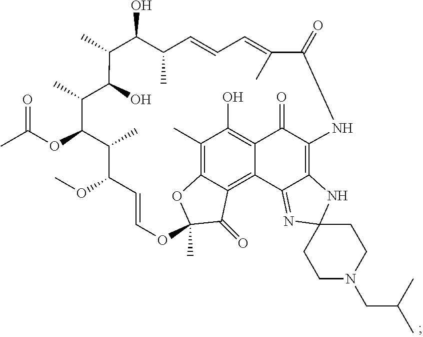 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(C)=O)[C@@H]1C)C4=O]

rifapentine, also known as (7S,9E,11S,12R,13S,14R,15R,16R,17S,18S,19E, 21Z,26E)-26-{[(4-cyclopentylpiperazin-1-yl)amino]methylidene}-2,15,17,29-tetrahydroxy-11-methoxy-3,7,12,14,16,18,22-heptamethyl-6,23,27-trioxo-8,30-dioxa-24-azatetracyclo [23.3.1.1^{4,7}.0^{5,28}]triaconta-1(28),2,4,9,19,21,25(29)-heptaen-13-yl acetate):

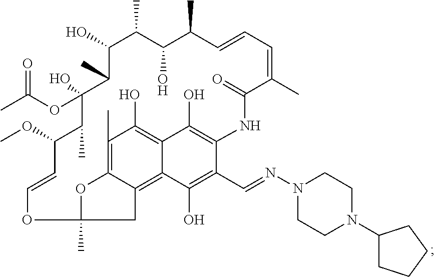 [SMILES: CO[C@H]1/C=C/O[C@]2(C)Cc3c(c(C)c(O)c4c(O)c(c(/C=N/N5CCN(C6CCCC6)CC5)c(O)c34)NC(=O)/C(C)=C\C=C\[C@H](C)[C@H](O)[C@@H](C)[C@@H](O)[C@@H](C)[C@](O)(OC(C)=O)[C@@H]1C)O2]

and,

rifaximin, also known as (2S,16Z,18E,20S,21S,22R,23R,24R,25S,26S,27S,28E)-5,6,21,23,25-pentahydroxy-27-methoxy-2,4,11,16,20,22,24,26-octamethyl-2,7-(epoxypentadeca-[1,11,13] trienimino)benzofuro[4,5-e]pyrido[1,2-a]-benzimida-zole-1,15(2H)-dione,25-acetate:

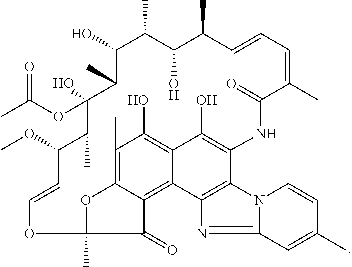

As used herein, the term “rifamycin-containing medication” refers to a medication comprising at least one member of the rifamycin class of drugs. In certain embodiments, the rifamycin-containing medication comprises at least one member of the rifamycin class of drugs as the single therapeutic agent.

By the term “specifically bind” or “specifically binds,” as used herein, is meant that a first molecule preferentially binds to a second molecule (e.g., a particular receptor or enzyme), but does not necessarily bind only to that second molecule.

By the term “specifically react” or “specifically reacts,” as used herein, is meant that a first molecule preferentially reacts with a second molecule (e.g., a particular drug, or metabolite or analogue thereof), but does not necessarily react only with that second molecule.

As used herein, the terms “subject”, “individual” and “patient” are used interchangeably to refer to a human or non-human mammal. Non-human mammals include, for example, livestock and pets, such as ovine, bovine, porcine, canine, feline and murine mammals. Preferably, the subject is human.

As used herein, the term “Tb” refers to tuberculosis.

The term “treat,” “treating” or “treatment,” as used herein, means reducing the frequency or severity with which symptoms of a disease or condition are experienced by a subject by virtue of administering an agent or compound to the subject.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C═CH. The term “homopropargylic” refers to a group exemplified by —CH₂CH₂—C═CH. The term “substituted propargylic” refers to a group exemplified by —CR₂—C═CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C═CR, wherein each occurrence of R is independently H, alkyl, substituted alkyl, alkenyl or substituted alkenyl, with the proviso that at least one R group is not hydrogen.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DISCLOSURE

The present invention relates to the unexpected discovery of a facile and versatile method of identifying a rifamycin, or a metabolite or analogue thereof, that is present in a biological sample obtained from a subject that is being administered a rifamycin-containing medication. In one aspect, the biological sample comprises urine.

The biological sample may be obtained from the subject that is being administered a rifamycin-containing medication using methods known to those skilled in the art. For example, urine of the subject may be collected at a given period of time after the subject is administered a rifamycin. In certain embodiments, the time between administration of the rifamycin-containing medication and collection of the sample is about 15 min, 30 min, 45 min, 1 h, 1 h 15 min, 1 h 30 min, 1 h 45 min, 2 h, 2 h 15 min, 2 h 30 min, 2 h 45 min, 3 h, 3 h 15 min, 3 h 30 min, 3 h 45 min, 4 h, 4 h 15 min, 4 h 30 min, 4 h 45 min, 5 h, 5 h 15 min, 5 h 30 mM, 5 h 45 min, 6 h, 6 h 15 min, 6 h 30 min, 6 h 45 min, 7 h, 7 h 15 min, 7 h 30 min, 7 h 45 min, 8 h, 8 h 15 min, 8 h 30 min, 8 h 45 min, 9 h, 9 h 15 min, 9 h 30 min, 9 h 45 min, 10 h, 10 h 15 min, 10 h 30 min, 10 h 45 min, 11 h, 11 h 15 min, 11 h 30 min, 11 h 45 min, 12 h, 14 h, 16 h, 18 h, 20 h, 22 h, 24 h, 30 h, 36 h, 42 h, 48 h, or any fraction or multiple amount thereof.

In certain embodiments, the biological sample is analyzed as isolated from the subject, i.e., it does not undergo any significant manipulation before it is used in the methods of the invention. In other embodiments, the biological sample is manipulated before it is used in the methods of the invention. The sample may also be manipulated, in non-limiting examples, to remove a compound that interferes with the methods of the invention, and/or to concentrate a rifamycin present in the sample, to render a rifamycin present in the biological sample available to be detected by the methods of the present invention (e.g., to free a rifamycin from a complex in which the rifamycin would not have been detected at all, or with lower sensitivity and/or selectivity, by the methods of the present invention). Such manipulation may be achieved by filtration, concentration, centrifugation, dilution with a liquid, fractional precipitation, extraction with a solvent, contacting with a solid (such as a resin comprising an immobilized reagent), and the like.

In a non-limiting example, the biological sample is extracted with an organic solvent, such as isoamyl alcohol, to extract or concentrate total rifamycin-related drugs from the biological sample. In another non-limiting example, the biological sample is subjected to chromatography or solid phase extraction using a simple modified silica gel cartridge to extract or concentrate total rifamycin-related drugs from the biological sample.

According to the invention, the biological sample, or a sample prepared by manipulation of the biological sample, may then be contacted with a derivatization reagent comprising an activated carboxylic acid, under conditions under which the activated carboxylic acid reacts with at least one phenolic group in the rifamycin in the sample.

In certain embodiments, the derivatization reagent comprises an UV-active and/or fluorescent group. In other embodiments, the UV-active and/or fluorescent group comprises at least one selected from the group consisting of coumarin, quantum dots (Xing, et al., 2007, Nature Protocols 2(5):1152, incorporated herein in its entirety), 6-(2,4-dinitrophenyl)aminohexanoic acid, the Alexa Fluor class of dyes (such as Alexa Fluor® 350, Alexa Fluor® 405, Alexa Fluor® 430, Alexa Fluor® 488, Alexa Fluor® 514, Alexa Fluor® 532, Alexa Fluor® 546 carboxylic acid), BODIPY class of dyes (such as 4,4-difluoro-5-(4-phenyl-1,3-butadienyl)-4-bora-3a,4a-diaza-s-indacene-3-propionic acid, BODIPY® 581/591, BODIPY® TMR-X, BODIPY® 493/503, and BODIPY® FL), Lissamine Rhodamine B, Malachite Green, Oregon Green 488, 5-(and-6) carboxynapthofluorescein, and PyMPO (1-(3-(succinimidyloxycarbonyl)benzyl)-4-(5-(4-methoxyphenyl)oxazol-2-yl)pyridinium bromide, available from Molecular Probes, Invitrogen, Life Technologies).

In certain embodiments, the derivatization reagent comprises at least one activated carboxylic acid selected from the group consisting of acyl chloride, symmetric or mixed acid anhydride, vinyl ester, cyanomethyl ester, S-phenyl thioester, piperidino ester, pyrid-3-yl ester, p-nitrophenyl ester, 2,4,6-trichlorophenyl ester, 2,3,4,5,6-pentachlorophenyl ester, 2,3,4,5,6-pentafluorophenyl ester, tetrafluorophenyl ester, phtalimido ester, succinimido ester, 4-oxo-3,4-dihydrobenzotriazin-3-yl ester, and benzotriazolyl ester. The derivatization reagents further comprises any solid supported version of the activated esters recited herein.

In certain embodiments, in the derivatization reagent the activated carboxylic acid is covalent connected to the UV-active and/or fluorescent group through a linker. In other embodiments, the linker comprises C₁-C₂₀ alkylene, C₃-C₂₀ cycloalkylene, (CH₂CH₂O)ₙ, (CH₂CH(CH₃)O)ₙ, an amino acid, or a peptide, wherein n=1-10, and the alkylene and cycloalkylene groups are independently optionally substituted with one or more substituents selected from the group consisting of C₁-C₆ alkyl, aryl, heteroaryl, heterocyclyl, hydroxyl, alkoxy and halo.

According to certain aspects of the invention, the activated carboxylic acid and the sample comprising a rifamycin are contacted, under conditions whereby the activated carboxylic acid reacts with at least one phenolic group in the rifamycin in the sample, thus forming an ester-comprising rifamycin derivative. In certain embodiments, the conditions used comprise incubation of an appropriately activated ester with the sample comprising a rifamycin under weakly basic conditions, such as in the presence of a weak organic base (e.g., triethylamine or diisopropylethylamine) or a weak inorganic base (e.g., an inorganic bicarbonate or carbonate). In certain embodiments, the activated carboxylic acid is immobilized on a solid support, whereby the rifamycin reacts with the solid-support immobilized activated carboxylic acid and generates a soluble ester-comprising rifamycin derivative.

Without wishing to be limited by any theory, the acidity and/or nucleophilicity of at least one the phenolic groups of the rifamycin allows for the selective and/or specific derivatization of the phenolic group in the presence of additional hydroxyl groups in the rifamycin or hydroxyl groups in other molecules present in the sample.

In certain embodiments of the invention, the ester-comprising rifamycin derivative is UV-active and/or fluorescent because the ester moiety therein comprises an UV-active and/or fluorescent group. In other embodiments of the invention, the ester-comprising rifamycin derivative is labeled with biotin or another affinity label that can be detected by affinity chromatography. In yet other embodiments of the invention, the ester-comprising rifamycin derivative is attached to a solid support and can be detected by affinity chromatography.

The ester-comprising rifamycin derivative may be detected using methods known to those skilled in the art.

In certain embodiments, the ester-comprising rifamycin derivative is submitted to a chromatographic separation process, whereby the derivative is eluted at a specific time under the separation conditions, thus allowing its detection. In other embodiments, the separation conditions are selected so that separate detection of a rifamycin derivative and the corresponding deacetylated rifamycin derivative is achieved.

In another non-limiting example, the ester-comprising rifamycin derivative is further captured by a solid phase particle, thereby being physically separated from the original solution in which the derivative was present. In certain embodiments, the immobilized derivative is subsequently released from the solid phase particle and detected by methods known to those skilled in the art.

In certain aspects of the invention, derivatized rifamycin and derivatized desacetyl rifamycin can be physically separated in a flow device due to differences in retention times, further allowing for the removal of non-drug related sample components. Detection of the rifamycin-dye conjugate (such as by UV and/or fluorescence detection) in a simple device allows of the quantification of the amount of the dye conjugate, and thus the amount of rifamycin present in the biological sample.

In certain aspects of the invention, the methods of the invention may be performed in a flow chemistry device. In other aspects, the device of the invention allows for the extraction of the total rifamycin, which is then incubated with supported activated ester for derivatization. The derivatized sample is then collected and analyzed in a simplified UV or fluorescence spectrophotometer for the desired UVₘₐₓ or fluorescence emission signals of conjugates of rifamycin and the desacetyl metabolite(s).

The compounds contemplated within the invention may possess one or more stereocenters, and each stereocenter may exist independently in either the (R) or (S) configuration. In certain embodiments, compounds described herein are present in optically active or racemic forms. The compounds described herein encompass racemic, optically-active, regioisomeric and stereoisomeric forms, or combinations thereof that possess the useful properties described herein.

In certain embodiments, the compounds contemplated within the invention may exist as tautomers. All tautomers are included within the scope of the compounds presented herein.

The methods and formulations described herein include the use of N-oxides (if appropriate), crystalline forms (also known as polymorphs), solvates, amorphous phases, and/or acceptable salts of compounds having the structure of any compound of the invention.

Solvates include water, ether (e.g., tetrahydrofuran, methyl tert-butyl ether) or alcohol (e.g., ethanol) solvates, acetates and the like. In certain embodiments, the compounds described herein exist in solvated forms with acceptable solvents such as water, and ethanol. In other embodiments, the compounds described herein exist in non-solvated form.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in analytical methods. In other embodiments, substitution with heavier isotopes such as deuterium affords greater chemical stability (for example, increased in vitro half-life). In yet other embodiments, substitution with positron emitting isotopes, such as ¹¹C, ¹⁸F, ¹⁵O and ¹³N, is useful in Positron Emission Topography (PET) studies for detecting the compounds. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds contemplated within the invention, such as derivatization reagents, described herein may form salts with acids, and such salts are included in the present invention. In certain embodiments, the salts are selected so that the compounds have appropriate crystallinity, stability to hydration and/or thermostability.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein. Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal. Protecting groups, plus a detailed description of techniques applicable to the creation of protecting groups and their removal are described in Greene & Wuts, Protective Groups in Organic Synthesis, 3rd Ed., John Wiley & Sons, New York, N Y, 1999, and Kocienski, Protective Groups, Thieme Verlag, New York, N.Y., 1994, which are incorporated herein by reference for such disclosure.

The compounds of the invention may be prepared according to the general methodology illustrated herein. The reagents and conditions described herein may be modified to allow the preparation of the compounds of the invention, and such modifications are known to those skilled in the art. The schemes included herein are intended to illustrate but not limit the chemistry and methodologies that one skilled in the art may use to make compounds of the invention.

The analytical assay used to detect the rifamycin, or metabolite or analogue thereof, may also be used to quantitate the concentration of the rifamycin, or metabolite or analogue thereof in the sample. In a typical procedure included in the invention, a series of standard solutions containing known concentrations of the rifamycin, or metabolite or analogue thereof, are prepared and analyzed using the methods of the invention. The readings obtained for each standard solution are used to create a calibration curve. The unknown sample is then analyzed by the same method, and its reading is compared to the standard curve in order to obtain a corresponding concentration of the rifamycin, or metabolite or analogue thereof, in the sample. This concentration may be used to calculate the actual concentration of the rifamycin, or metabolite or analogue thereof, in the biological fluid, taking into account the dilutions to which the biological sample was subjected. Use of the calibration curve, as described above, allows the concentration of the rifamycin, or metabolite or analogue thereof, to be determined in the same units used to express the concentration of the standard solutions. In some instances, the standard solutions have their component concentrations identified in mass/volume units (such as μg/L units, for example). The concentration of the rifamycin, or metabolite or analogue thereof, in the biological sample, determined for example as μg/L or μg/L from the calibration curve, may be converted to a concentration of moles/volume (such as nmol/L or μmol/L) based on the molecular weight of the rifamycin, or metabolite or analogue thereof.

The skilled artisan would appreciate, based on the present disclosure, that a method of the invention may be modified based on the needs of a particular application. By way of a non-limiting example, a particular reagent may be added to a particular analyte solution if the reagent does not have the potential to interfere with the assay or with one or more other components of the analyte solution. By way of another non-limiting example, a particular reagent is not added to a particular analyte solution if the reagent has the potential to interfere with the assay or with one or more other components of the analyte solution. Similarly, the skilled artisan will know, based on the present disclosure, that a method of the invention may be modified based on the needs of a particular application. By way of a non-limiting example, the assay may utilize an endpoint reaction, wherein the concentration of the rifamycin, or metabolite or analogue thereof, is measured at equilibrium. By the way of another non-limiting example, the assay may measure the rate of formation of the rifamycin, or metabolite or analogue thereof, over a period of time or at one or more time points.

The skilled artisan would further appreciate, based upon the disclosures provided herein, that the invention is not limited to any particular instrument, but rather the invention encompasses a wide plethora of instruments as are known in the art or to be developed in the future. That is, such instruments for assessing the presence and/or level of a known constituent of interest in a sample include, but are not limited to, a UV spectrophotometer and/or fluorescence spectrophotometer. Thus, the skilled artisan would understand, based upon the disclosure provided herein, that the invention is not limited in any way to any particular instrument, either known or to be developed. Such instruments, including hand-held devices, single test devices, and the like, are well-known in the art.

As will be understood by one of skill in the art, when armed with the disclosure set forth herein, a set of at least one reference rifamycin, or metabolite or analogue thereof (also referred to as “calibration samples”), may be used to create a calibration curve for a certain method and/or instrument. By way of a non-limiting example, the set of at least one reference rifamycin, or metabolite or analogue thereof may be used in a two-point calibration assay. In another embodiment of the invention, the set of at least one reference rifamycin, or metabolite or analogue thereof may be used in a five- or six point calibration assay. In one aspect, the set of at least one reference rifamycin, or metabolite or analogue thereof may include as many or as few reference points as determined to be necessary to establish a valid and accurate reference curve.

Numerous calibration schemes may be used in the clinical laboratory. Older methods, often manually performed, employ several concentration levels throughout the assay range and typically plot the instrumental response versus concentration or use linear regression to calculate patient analyte values. These methods may still be used. However, with the increasing use and availability of computer technology, methods now often use one or two calibrator points to achieve the same results. Quite often, the one or two set point method incorporates a saline or distilled water blank as an additional set point, this latter function being dictated by the instrument or reagent manufacturer. For non-linear chemistries, the traditional approach provides five or six levels of calibrator, usually set in a non-linear fashion dictated by the mathematical model used in the final calculation of patient result. A more recent trend for non-linear chemistries is to use one calibrator containing the highest concentration of analyte measured in the assay. Using this method, the analytical system is then directed to perform the necessary dilutions of this high concentration value to generate the predetermined calibration set points on the fly when the system calibrates the analyte. A four- or five-parameter logit/log calibration curve is typically used for automated immunoassays.

Therefore, in an aspect of the present invention, there is provided a method that features the use of multiple calibrator points in order to generate a reference curve. In certain embodiments, the method features the use of more than one point. In other embodiments, one of the multiple points is a zero point. In yet other embodiments, the zero point is not included as one of the multiple points, but may be included separately in a reference curve. In other embodiments, the method features the use of a single calibration point, as described in detail elsewhere herein. In yet other embodiments, the method features the use of a zero point in addition to a single calibration point.

By way of a series of non-limiting examples, the method of the invention may use a reference curve based on a single concentration for calibration, a reference curve based on a single concentration plus a zero concentration point for calibration, a reference curve based on at least two concentrations for calibration, or a reference curve based on at least two concentrations plus a zero concentration point for calibration. In one embodiment of the invention, the concentration of a calibration sample is known. In another embodiment of the invention, the concentration of a calibration sample is not known. In yet another embodiment of the invention, the concentration of at least one calibration sample in a mixture containing at least two calibration samples is known.

### Kits

The invention includes various kits that comprise a set of at least one rifamycin, or metabolite or analogue thereof (“calibration samples”), an applicator, and instructional materials that describe use of the kit to perform the methods of the invention. Although exemplary kits are described below, the contents of other useful kits will be apparent to the skilled artisan in light of the present disclosure. Each of these kits is included within the invention.

In certain embodiments, the invention includes a kit for measuring the concentration of at least one rifamycin drug, or metabolite or analogue thereof, in a biological sample of a subject. The kit comprises reagents that allow for the determination of at least one rifamycin, or metabolite or analogue thereof. The kit further comprises an applicator and instructional material for the use of the kit.

The kit is used pursuant to the methods disclosed in the invention. In certain embodiments, the kit may be used to determine the concentration of at least one rifamycin, or metabolite or analogue thereof, in a biological sample. This is because, as more fully disclosed elsewhere herein, the data disclosed herein demonstrates that the derivatization reaction allows for the detection of a rifamycin and the corresponding deacetylated product(s).

The kit further comprises an applicator useful for administering the reagents for use in the relevant assay. The particular applicator included in the kit will depend on, e.g., the method used to assay at least one rifamycin, or metabolite or analogue thereof, as well as the particular analyzer equipment used, and such applicators are well-known in the art and may include, among other things, a pipette, a syringe, a dropper bottle, and the like. Moreover, the kit comprises an instructional material for the use of the kit.

Further, the kit includes a kit comprising at least one reference composition comprising a known value of a known constituent, which may be a rifamycin, or metabolite or analogue thereof. Such kits may be used to create a calibration curve for quantitation of a rifamycin, or metabolite or analogue thereof. Thus, the invention encompasses a kit comprising at least one reference composition. While the invention is not limited to any particular set, certain combinations of reference compositions are exemplified elsewhere herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all remaining starting materials were obtained from commercial suppliers and used without purification.

### Example 1: Acylation of Rifampicin

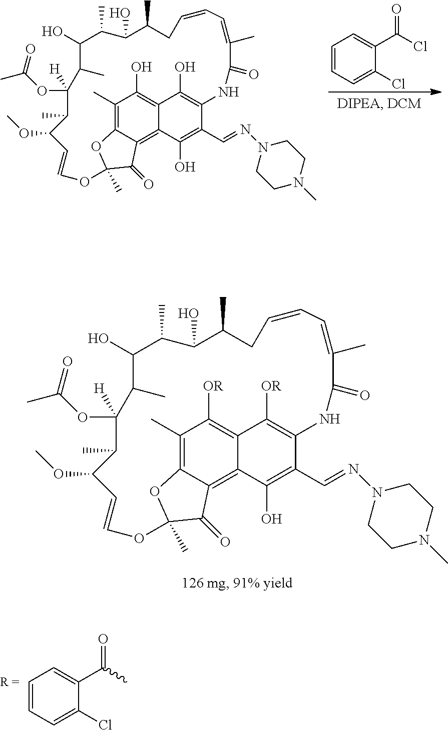

To a stirred solution of rifampicin (100 mg, 0.12 mmol) in dry DCM (3.0 mL) was added DIPEA (64 μL, 0.36 mmol), and 2-chlorobenzoyl chloride (32 μL, 0.25 mmol) was added dropwise to this mixture under room temperature. The reaction mixture was stirred until rifampicin was consumed (as monitored by TLC analysis, 30 min).

The mixture was poured into water and washed with DCM. The organic phase was dried with anhydrous Na₂SO₄ and evaporated to dryness. The red residue was purified by flash chromatography on silica gel (DCM/MeOH=50/1) to yield the product as a red solid (126 mg, 91% yield). The compound was characterized by ¹H NMR and the spectral data were consistent with the structure.

### Example 2: Acylation of Rifampicin

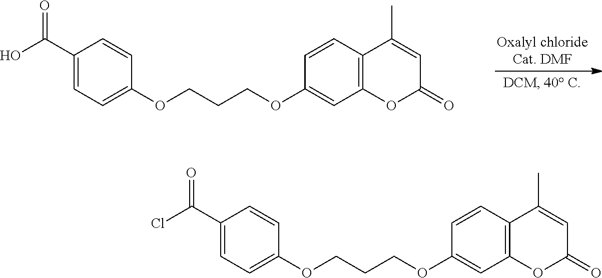 [SMILES: Cc1cc(=O)oc2cc(OCCCOc3ccc(C(=O)O)cc3)ccc12 ; Cc1cc(=O)oc2cc(OCCCOc3ccc(C(=O)Cl)cc3)ccc12]

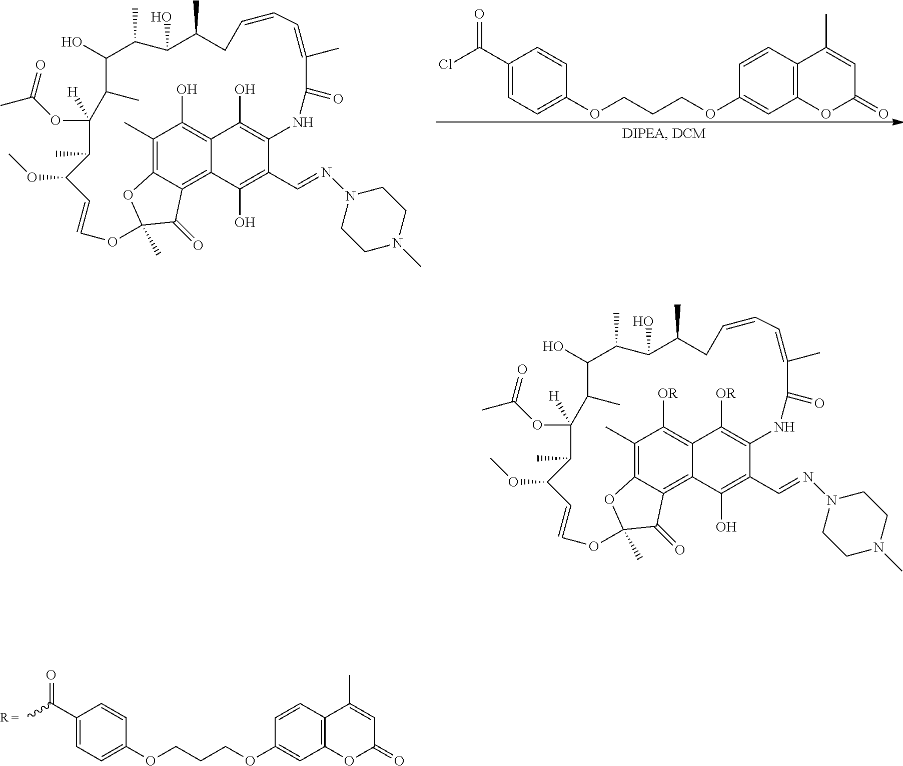

To a stirred solution of 4-[3-(4-methyl-2-oxo-2H-chromen-7-yloxy)-propoxy]-benzoic acid (160 mg, 0.45 mmol) in dry DCM (3.0 mL) was added DMF (7 μL, 0.09 mmol), and oxalyl chloride (46 μL, 0.55 mmol) was added dropwise to this mixture under room temperature. The reaction mixture was stirred at 40° C. for 5 hours. DCM was removed in vacuum, yielding crude 4-[3-(4-methyl-2-oxo-2H-chromen-7-yloxy)-propoxy]-benzoyl chloride.

To a stirred solution of rifampicin (150 mg, 0.18 mmol) in dry DCM (3.0 mL) was added DIPEA (96 μL, 0.54 mmol), and 4-[3-(4-methyl-2-oxo-2H-chromen-7-yloxy)-propoxy]-benzoyl chloride in 2 mL DCM was added dropwise to this mixture at room temperature. The reaction mixture was stirred for 1 hour, poured into water and washed with DCM. The organic phase was dried with anhydrous Na₂SO₄ and evaporated to dryness. The red residue was purified by flash chromatography on silica gel (DCM/MeOH=50/1) to yield the product as a red solid (63 mg, 42% yield). The compound was characterized by ¹H NMR and the spectral data were consistent with the structure.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of detecting the presence of a rifamycin, or metabolite or analogue thereof, in a sample, the method comprising the steps of:
contacting the sample with a derivatization reagent comprising an activated carboxylic acid, using conditions under which at least one phenolic group in the rifamycin, or metabolite or analogue thereof, reacts with the activated carboxylic acid to form an ester-comprising rifamycin derivative, thereby generating a reaction mixture; and,
analyzing the reaction mixture for the presence of the ester-comprising rifamycin derivative;
wherein, if the derivative is present in the reaction mixture, the rifamycin, or metabolite or analogue thereof, is detected in the sample.

2. The method of claim 1, wherein the sample comprises a biological sample from a subject that is administered a rifamycin-containing medication.

3. (canceled)

4. The method of claim 1, wherein the derivatization reagent comprises a chromophore.

5. The method of claim 4, wherein the chromophore is UV-active or fluorescent.

6. The method of claim 2, wherein the rifamycin-containing medication comprises at least one selected from the group consisting of rifampicin, rifamycin B, rifamycin SV, rifamycin S, rifabutin, rifapentine, rifaximin, and metabolites and analogues thereof.

7. The method of claim 2, wherein the rifamycin is present in the rifamycin-containing medication or the rifamycin metabolite comprises the deacetylated derivative of the rifamycin-containing medication.

8. (canceled)

9. The method of claim 2, wherein the sample is obtained from the subject at a given period of time after the subject is administered the rifamycin-containing medication.

10. The method of claim 1, wherein the activated carboxylic acid is selected from the group consisting of acyl chloride, symmetric or mixed acid anhydride, vinyl ester, cyanomethyl ester, S-phenyl thioester, piperidino ester, pyrid-3-yl ester, p-nitrophenyl ester, 2,4,6-trichlorophenyl ester, 2,3,4,5,6-pentachlorophenyl ester, tetrafluorophneyl ester, 2,3,4,5,6-pentafluorophenyl ester, phtalimido ester, succinimido ester, 4-oxo-3,4-dihydrobenzotriazin-3-yl ester, and benzotriazolyl ester.

11. The method of claim 1, wherein the derivatization reagent is immobilized on a solid support.

12. The method of claim 1, further comprising the steps of:
contacting the derivatization reagent with at least one control sample comprising a known amount of the rifamycin, or metabolite or analogue thereof; thereby generating a control mixture; and,
analyzing the control mixture for the presence of the ester-comprising rifamycin derivative.

13. The method of claim 11, wherein the analysis of the control mixture allows for the generation of a calibration curve for the rifamycin, or metabolite or analogue thereof.

14. The method of claim 12, further comprising the step of quantitating the amount or concentration of the rifamycin, or metabolite or analogue thereof, in the sample.

15. (canceled)

16. A method of determining whether a subject is being administered an effective dosage of a rifamycin-containing medication, the method comprising the steps of:
contacting a biological sample from the subject with a derivatization reagent comprising an activated carboxylic acid, using conditions under which at least one phenolic group in the rifamycin, or metabolite or analogue thereof, reacts with the activated carboxylic acid to form an ester-comprising rifamycin derivative, thereby generating a reaction mixture;
analyzing the reaction mixture for the presence of the ester-comprising rifamycin derivative;
determining the concentration of the rifamycin, or metabolite or analogue thereof in the biological sample; and,
determining the circulating concentration of the rifamycin, or metabolite or analogue thereof, in the subject;
thereby determining whether the subject is being administered an effective dosage of the rifamycin-containing medication.

17. The method of claim 16, further comprising the step of adjusting the subject's dosage of the rifamycin-containing medication so that a therapeutically effective rifamycin circulating concentration is reached in the subject.

18. (canceled)

19. The method of claim 16, wherein the derivatization reagent comprises a chromophore.

20. The method of claim 19, wherein the chromophore is UV-active or fluorescent.

21. The method of claim 16, wherein the rifamycin-containing medication comprises at least one selected from the group consisting of rifampicin, rifamycin B, rifamycin SV, rifamycin S, rifabutin, rifapentine, rifaximin, and metabolites and analogues thereof.

22. The method of claim 16, wherein the rifamycin is present in the rifamycin-containing medication or the rifamycin metabolite comprises the deacetylated derivative of the rifamycin-containing medication.

23. (canceled)

24. The method of claim 16, wherein the sample is obtained from the subject at a given period of time after the subject is administered the rifamycin-containing medication.

25. (canceled)

26. A kit comprising an activated carboxylic acid, wherein the activated carboxylic acid reacts with at least one phenolic group in a rifamycin, or metabolite or analogue thereof, and instructional material comprising instructions how to detect the rifamycin, or metabolite or analogue thereof, using the activated carboxylic acid as a reagent.
